(19) United States
(12) Patent Application Publication
(10) Pub. No.: US 2013/0281761 A1
(43) Pub. Date: Oct. 24, 2013

(54) CANNULA WITH BIFURCATED TIP FOR A CARDIAC ASSIST DEVICE

(71) Applicant: Navin K. Kapur, Hanover, MA (US)

(72) Inventor: Navin K. Kapur, Hanover, MA (US)

(73) Assignee: Tufts Medical Center, Inc., Boston, MA (US)

(21) Appl. No.: 13/883,053

(22) PCT Filed: Oct. 31, 2011
(86) PCT No.: PCT/US11/58540

§ 371 Date: Jul. 11, 2013

Related U.S. Application Data
(60) Provisional application No. 61/410,431, filed on Nov. 5, 2010.

(51) Int. Cl. A61M 1/10

(52) U.S. Cl. CPC A61M 1/1086; A61M 1/101
USPC 600/17; 600/16

(57) ABSTRACT

A cardiac assist device includes a cannula that terminates in a flexible tip. The tip is generally Y-shaped and includes a proximal end that extends from the end of the cannula, and a bifurcated distal end opposed to the proximal end. The bifurcated distal end includes a first portion detached from a second portion, and the tip further includes a pair of through channels extending from the proximal end to the bifurcated distal end. The tip is configured so that one channel of the pair of channels extends through the first portion, and the other channel of the pair of channels extends through the second portion.

### CROSS REFERENCE TO RELATED APPLICATIONS

This application claims the priority of U.S. provisional application No. 61/410,431 filed on Nov. 5, 2010, the contents of which are incorporated by reference in their entirety.

### BACKGROUND OF THE INVENTION

The incidence of cardiogenic shock following acute myocardial infarction (AMI) is 8.6%. The right ventricle (RV) is involved in greater than one-third of all inferior myocardial infarctions (MI). Mortality after RVMI approaches 60% and is a major global healthcare concern.

The incidence of cardiogenic shock following acute myocardial infarction (AMI) is 8.6%. The right ventricle (RV) is involved in greater than one-third of all inferior myocardial infarctions (MI). Mortality after RVMI approaches 60% and is a major global healthcare concern.

Management of right heart failure secondary to any cause conventionally includes one or more of fluid resuscitation, vasopressor and inotropic support, and trans-venous pacing in the setting of high-grade atrio-ventricular conduction block. Historically, mechanical support for RV infarction has been limited to intra-aortic balloon pump (IABP) counterpulsation or surgically placed ventricular assist devices. Percutaneously implanted RV assist devices (pRVAD) offer an intermediate alternative for patients with refractory right heart failure in the setting of AMI. The standard approach to pRVAD cannulation is via the femoral vein and artery.

### SUMMARY

In one aspect, a flexible tip is provided that is configured to extend from an end of a cannula. The tip includes a proximal end that extends from the end of the cannula, and a bifurcated distal end opposed to the proximal end.

The tip may include one or more of the following features: The tip is generally Y shaped. The tip further includes a pair of through channels extending from the proximal end to the bifurcated distal end, the bifurcated distal end includes a first portion detached from a second portion. One channel of the pair of channels extends through the first portion, and the other channel of the pair of channels extends through the second portion. Each channel is configured to receive a guide wire therethrough. The tip is configured to permit adjustment of the distance of the distal end from the end of the cardiac assist device. The tip includes fluid pressure sensors. The cannula is a housing for a percutaneous cardiac assist device that is configured to be disposed at least partially within the heart when in use, and the tip is configured to extend from an end of the percutaneous cardiac assist device. The bifurcated distal end includes a first tip portion and a second tip portion that is detached from the first tip portion, and wherein each of the first tip portion and the second tip portion is configured to curl back on itself. An other end of the cannula is connected to a cardiac assist device, the cardiac assist device configured to reside outside the body when in use.

In another aspect, a percutaneous cardiac assist device is provided. The device includes a fluid pump, a tube configured to provide a passageway for fluid pumped by the fluid pump, and a bifurcated, flexible tip. The tube includes a tube first end, and a tube second end opposed to the tube first end, wherein the tube second end is configured to serve as a fluid outlet from the pump, and the bifurcated, flexible extends from the tube second end.

The device may include one or more of the following features: The first tube end is configured to serve as an inlet to the fluid pump. The tip includes a proximal end configured to secure to the tube second end, a bifurcated distal end opposed to the proximal end and including first tip portion and a second tip portion that is detached from the first tip portion; a first channel extending through the tip between the proximal end and a terminal end of the first tip portion, the first channel configured to receive a guide wire; and a second channel extending through the tip between the proximal end and a terminal end of the second tip portion, the second channel configured to receive a guide wire. Each of the first tip portion and the second tip portion is configured to curl back on itself when a guide wire is not present within the respective first and second channel. The tip is generally Y shaped. The cardiac assist device is configured to be deployed to the heart via at least one of the superior vena cava and the inferior vena cava. The cardiac assist device is configured to be deployed to the heart via the jugular vein. The distance of the distal end from the tube second end is adjustable. The tip includes fluid pressure sensors. The fluid pressure sensors are disposed in the proximal end of the tip. The tube includes a lumen that is in fluid communication with the first and second channels.

In another aspect, a method of using a percutaneous assist device having a dual-lumened flexible tip is disclosed. The leading end of the tip is bifurcated to form a first tip portion and a second tip portion that is detached from the first tip portion, each of the first and second tip portions including a respective lumen. The method including the steps of forming a percutaneous puncture in the jugular vein; advancing a first guide wire through the puncture to the right pulmonary artery; advancing a second guide wire through the puncture to the left pulmonary artery; mounting the assist device on both the first and second guide wires such that the first guide wire extends through one respective lumen and the second guide wire extends through the other respective lumen; advancing the assist device along the first and second guide wires until the first tip portion resides in the right pulmonary artery, and the second tip portion resides in the left pulmonary artery; and withdrawing the guide wires from respective the lumens to permit the first and second tip portions to support the assist device within the pulmonary artery. The method may also include the step of providing treatment fluids to the body through at least one of the lumens.

The leading end of the percutaneous cardiac assist device (pCAD) advantageously includes a bifurcated tip which supports the device and maintains the proper position of the device within a branched vessel of the body. For example, when the pCAD is used to provide right ventricular support, the bifurcated tip includes a first portion that is placed within right pulmonary artery and a second portion that is placed within the left pulmonary artery, whereby the assist device is maintained in the main (unbranched portion) pulmonary artery. The bifurcated tip tip allows for equal distribution of blood flow into both lung fields and prevents the device from migrating into either the right or left lung. Such antegrade migration or selective lung perfusion can cause harm to patients by inducing pulmonary hemorrhage or heart failure. Thus, the bifurcated tip enhances secure placement of the device in the main pulmonary artery by avoiding antegrade migration into the lungs.

In addition, by including pressure sensors in the bifurcated tip, improved hemodynamic monitoring of heart function during support and weaning is achieved. Furthermore, modification of the bifurcated tip can allow for delivery of pharmacologic agents into selective lung fields. This may be particularly helpful in clinical situations where 1) thrombolytic therapy is required to dissolve a thrombotically occluded pulmonary artery (a major cause of right heart failure), 2) selective pulmonary vasodilator therapy is necessary, or 3) if patients have limited vascular access and medications need to be administered systemically.

A method is described that allows for percutaneous placement of the bifurcated cannula via the jugular or subclavian veins. Approach from these locations is advantageous since it allows for improved patient mobility resulting in faster recovery times and reduced likelihood of infection with the device in place. Furthermore, approaching the pulmonary artery from these locations is technically less complicated as the catheter follows the natural curvature of the right-sided circulation. This is in opposition to the femoral approach, which requires more mechanical manipulation for cannula placement.

Modes for carrying out the present invention are explained below by reference to an embodiment of the present invention shown in the attached drawings. The above-mentioned object, other objects, characteristics and advantages of the present invention will become apparent from the detailed description of the embodiment of the invention presented below in conjunction with the attached drawings.

### BRIEF DESCRIPTION OF THE DRAWINGS

FIG. 1 is a diagrammatic view of the percutaneous cardiac assist device in the human body.

FIG. 2 is a top plan view of the device of FIG. 1.

FIG. 3 is a sectional view of the device as seen along line 3-3 of FIG. 2.

FIG. 4 is a sectional view of the device as seen along line 4-4 of FIG. 2.

FIG. 5 is a sectional view of the device as seen along line 5-5 of FIG. 2.

FIG. 6 is a sectional view of the device as seen along line 6-6 of FIG. 2.

FIGS. 7-13 illustrate the method of using the device using an intra jugular approach.

FIG. 14 is an alternative embodiment of the device.

### DETAILED DESCRIPTION

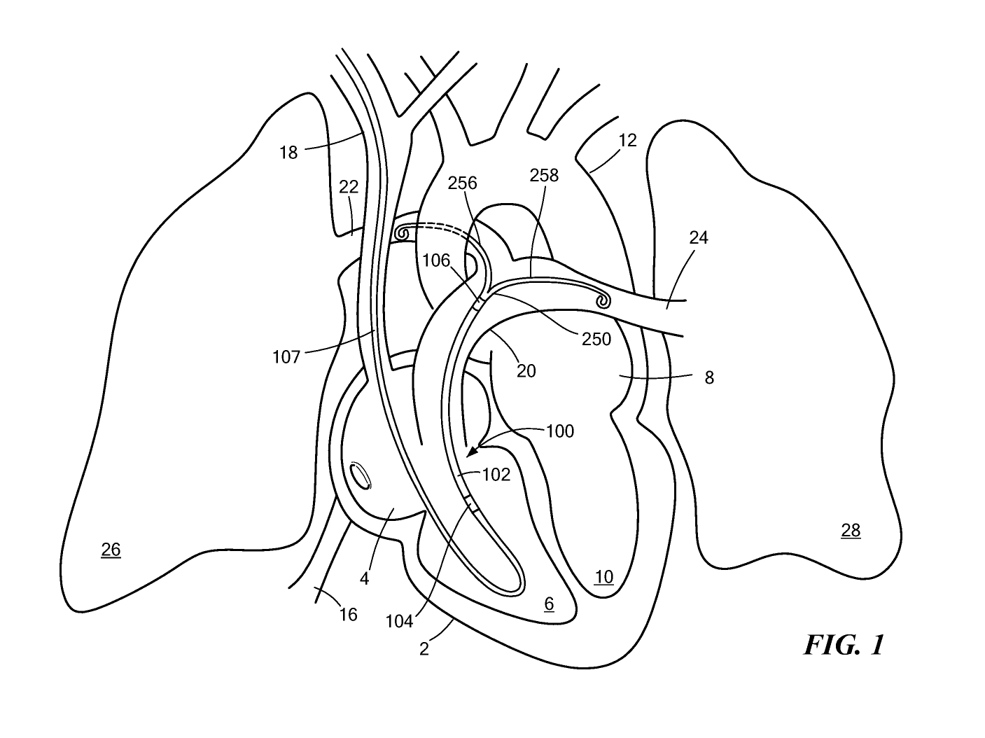

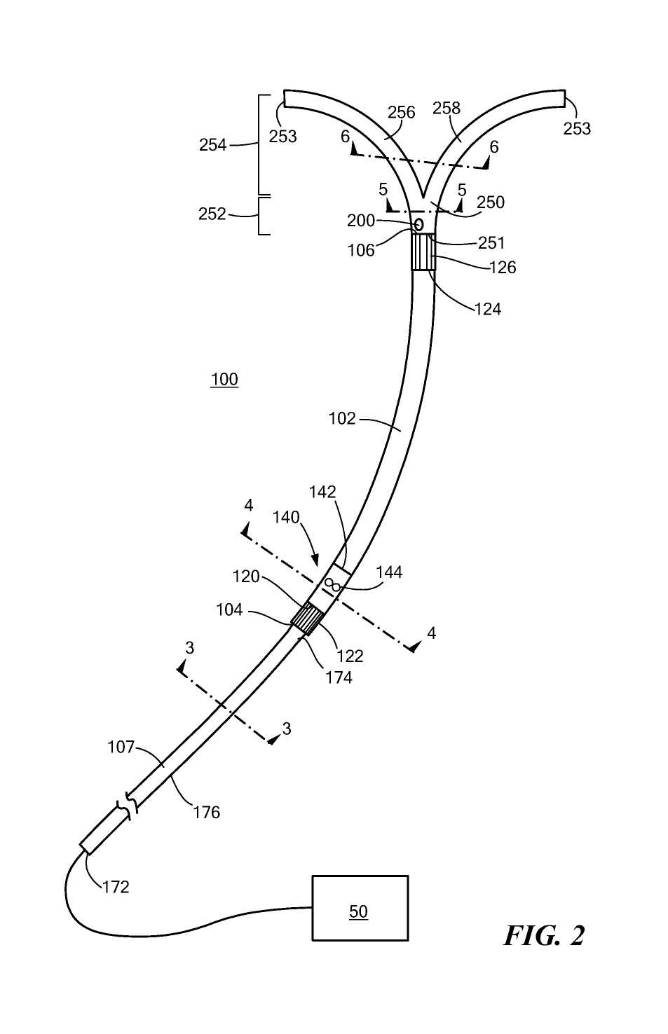

Referring now to FIGS. 1 and 2, a percutaneous cardiac assist device (pCAD) 100 may be positioned within a heart 2 so that an inlet end 104 of the device is located in the right ventricle 6 and the outlet end 106 is located in the main pulmonary artery 20. The pCAD 100 includes a fluid pump 140 supported within a flexible cylindrical cannula 102 that serves as a device housing. The pump 140 draws blood of the right ventricle 6 into the inlet end 104 of the cannula 102 and expels it from the outlet end 106 into the main pulmonary artery 20. The inlet and outlet ends 104, 106 of the cannula 102 are provided with wire cages 122, 126 that permit free flow of blood into or out from the respective end, while preventing damage to adjacent vessel tissues. The device 100 includes a catheter 170 that is joined to the inlet end 104 of the cannula 102, and a flexible bifurcated tip 250 that is disposed on the outlet end 106. The bifurcated tip 250 serves to secure placement of the device 100 in the pulmonary artery 20, as discussed further below.

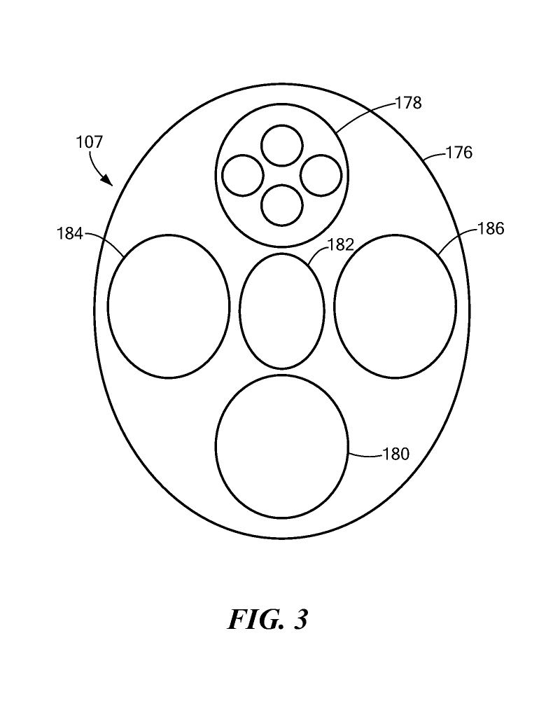

Referring to FIGS. 2 and 3, the catheter 107 includes an elongated tubular housing 176 having a length sufficient to extend from the device cannula 102, through the heart 2 and blood vessels to a controller and power supply 50 located externally of the body. In the illustrated embodiment, the catheter 107 is a 12-14 French catheter and includes lumens that extend between opposed ends 172, 174, providing a passageway for delivering devices and fluids between the device 100 and the exterior of the body. For example, the catheter 107 includes a wiring lumen 178 which holds electrical leads for operating and controlling the pump 140, an open central lumen 182, and sensor fluid lumen 180 which provides fluid to the pressure sensors 200. The catheter 107 also includes first and second open lumens 184, 186 which communicate with corresponding passageways provided in the device tip 250, as discussed further below. The first and second open lumens 184, 186 are each sized to accommodate a guide wire and are capable of providing drug delivery to the device tip 250.

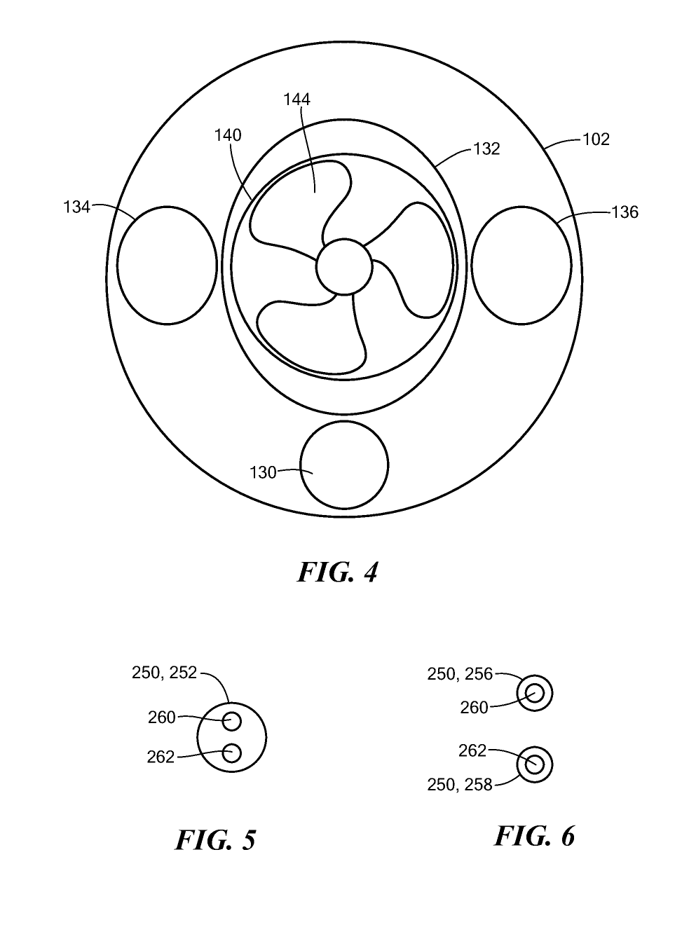

Referring to FIGS. 2 and 4, the cannula (device housing) 102 is slightly larger in diameter than the catheter 107 so as to accommodate the fluid pump. For example, in the illustrated embodiment the cannula 102 is a 22 French tube and includes lumens that extend between opposed ends 104, 106, providing a passageway for delivering devices and fluids between the respective cannula ends 104, 106. The cannula 102 includes a relatively large central lumen 132 sized to accommodate the fluid pump 140 disposed therein, and to provide a passageway for blood drawn through the cannula 102. The cannula 102 also includes additional lumens which are small in diameter relative to the central luman 132. In particular, the cannula 102 includes a sensor fluid lumen 130 that communicates with the corresponding catheter sensor fluid lumen 180 and provides fluid to the pressure sensors 200. In addition, the cannula 102 includes first and second open lumens 134, 136 which connect corresponding passageways provided in the catheter 107 and the device tip 250. Specifically, the first open lumen 134 connects the first catheter open lumen 184 with the device tip first channel 260 (discussed further below), and the second open lumen 136 connects the second catheter open lumen 186 with the device tip second channel 262 (discussed further below). The cannula 102 also includes a wiring lumen (not shown) which joins the catheter wiring lumen 178 and the fluid pump 140, and thus does not extend along the full length of the cannula 102.

The tip 250 is flexible, elastic member disposed on the outlet end of the device 100. The tip 250 is generally Y-shaped and includes a main portion 252 connected to outlet end 106 the device 100, and a bifurcated portion 254 extending from the main portion 252. In the illustrated embodiment, the bifiurcated portion 254 is much longer than the main portion 252. For example, the bifurcated portion 254 may provide 60 to 90 percent of the overall length of the tip 250. In addition, bifurcated portion 254 may be more flexible than the main portion 252.

The main portion 252 of the tip 250 includes a tip proximal end 251 that is connected to the outlet cage 126 of the device 100 by conventional means. The bifurcated portion 254 that extends from the main portion 252 includes a first tip portion 256 and a second tip portion 258. The first and second tip portions 256, 258 are separated from each other and terminate in respective distal ends 253.

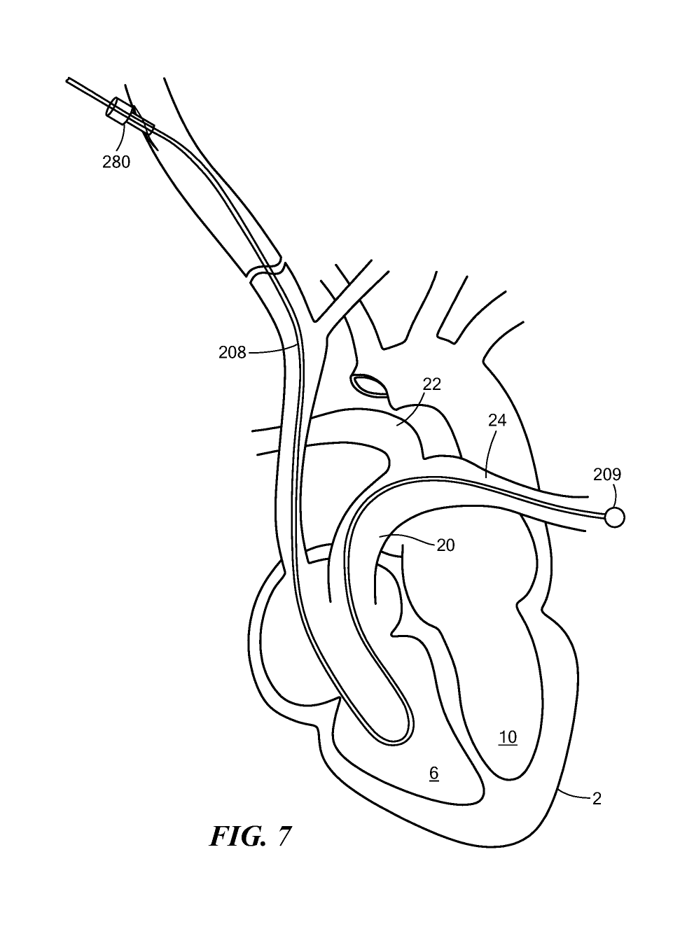

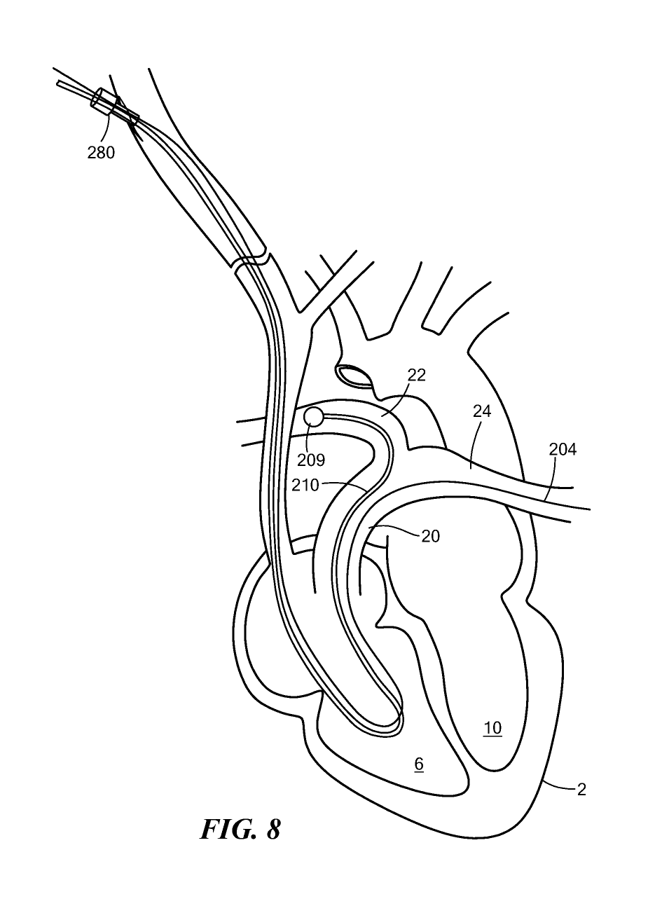

Referring also to FIGS. 5 and 6, the tip 250 further includes a pair of through channels 260, 262 extending from the proximal end 251 to the distal end 253. Each channel 260, 262 is configured to receive a guide wire and permit delivery of therapeutic agents therethrough. The first channel 260 of the pair of channels extends through the first portion 256, and is configured to communicate with the first open lumen 134 of the cannula 102. Similarly, the second channel 262 of the pair of channels extends through the second portion 258, and is configured to communicate with the second open lumen 136 of the cannula 102.

Each of the first and second tip portions 256, 258 is sufficiently flexible and elastic to conform to the shape of a guide wire disposed within the respective channel 260, 262 and to curl back on itself when the guide wire is removed from the device 100. In addition, the each of the first and second tip portions 256, 258 is sufficiently rigid to support and secure the device in a desired location within the blood vessel, as discussed further below.

The main portion 252 includes fluid pressure sensors 200 disposed adjacent the proximal end 251. The fluid pressure sensors 200 are connected to the sensor fluid lumen 130 of the cannula 102, whereby detected information corresponding to vessel pressures at this location can be relayed to the controller 50 via the cannula 102 and catheter 107.

The cannula 102 is provided having a length that permits the cannula 102 to be disposed at least partially within the heart 2 when in use. More specifically, when in use, the inlet end 104 of the housing is disposed within the right ventricle 6 of the heart 2 and the outlet end 106 of the cannula 102 is disposed within the main pulmonary artery 20. In addition, the first portion 256 of the tip 250 is positioned in the right pulmonary artery 22, and the second portion 258 of the tip 250 is positioned in the left pulmonary artery 24. By this arrangement, the bifurcated portion 254 straddles artery branches 22, 24, the device 100 is prevented from moving into either the right or left pulmonary arteries 22, 24, and instead is maintained in the desired location within the main pulmonary artery 20.

Referring to FIGS. 7 to 13, a method of using the percutaneous assist device 100 having the dual-lumened flexible tip 250 in an intra jugular approach will now be described.

Referring to FIG. 7, a percutaneous puncture is formed in the jugular vein (not shown). A ballooned and steerable insertion catheter 208 is inserted into the puncture through a vascular sheath 280, for example a right intra jugular vascular sheath, and the balloon 209 of the insertion catheter inflated. As a result the insertion catheter 208 is drawn through blood vessels from the incision site, through the heart 2, through the main pulmonary artery 20, to a first branch of the pulmonary artery 20. In this example, the insertion catheter is directed to the left pulmonary artery 24.

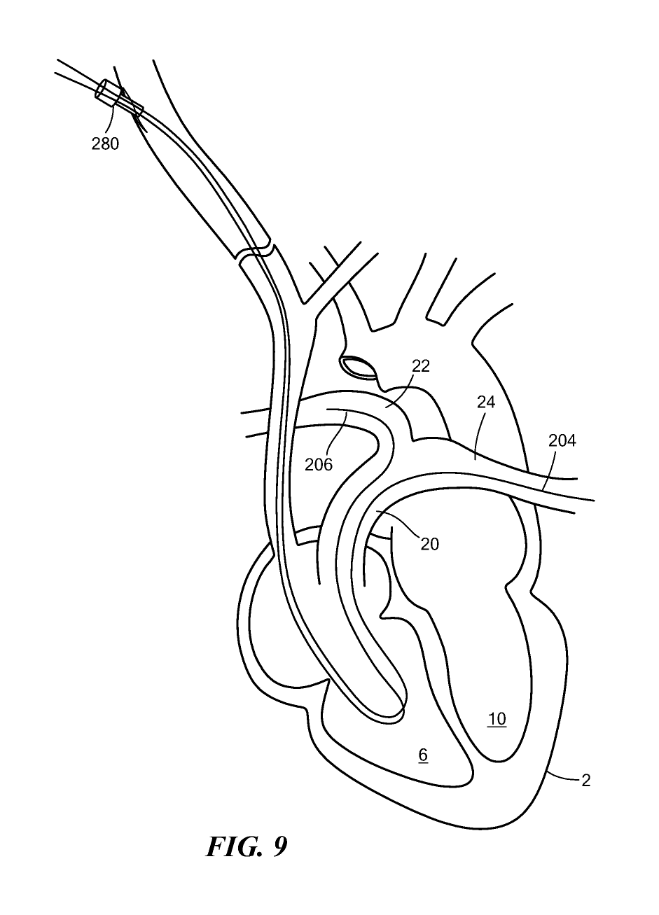

Referring to FIG. 8, a first guide wire 204 is advanced through the insertion catheter 208 to the left pulmonary artery 24, and then the insertion catheter 208 is removed leaving the first guide wire 204 in place in the left pulmonary artery 24. A second insertion catheter 210 is inserted into the vein through the same vascular sheath, and allowed to travel to the other branch of the pulmonary artery 20, in this example the right pulmonary artery 22 as described above, and a second guide wire 206 is advanced through the insertion catheter 210 to the right pulmonary artery 22.

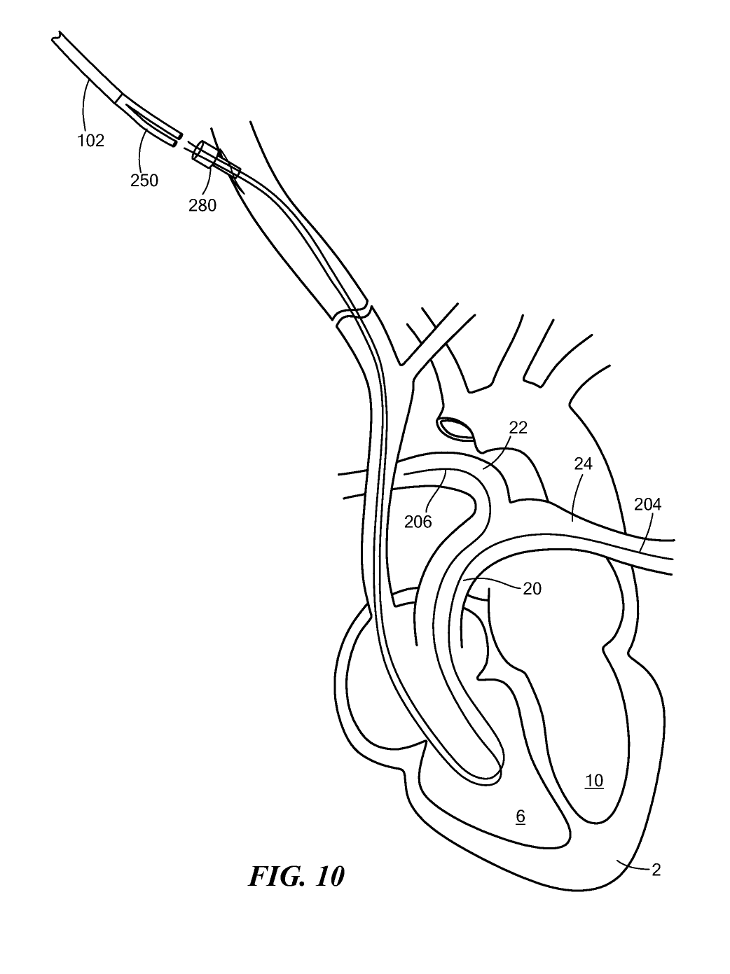

Referring to FIG. 9, the insertion catheter 210 is removed, leaving the second guide wire 206 in place in the right pulmonary artery 22.

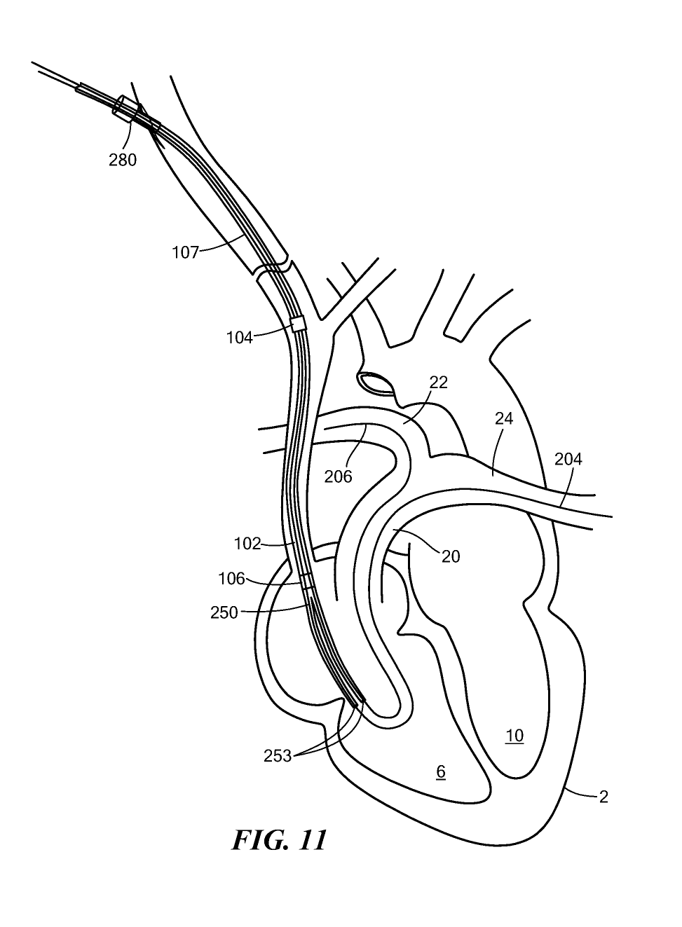

Referring to FIGS. 10 and 11, once the guide wires 206, 208 are in place in respective branches of the pulmonary artery 20, the pCAD 100 is loaded onto the guide wires 204, 206 such that the first guide wire 204 extends through the continuous passageway formed by the first tip channel 260, the first open lumen 134 of the device cannula 102, and the first open lumen of the catheter 107, and the second guide wire 204 extends through the continuous passageway formed by the second tip channel 262, the second open lumen 136 of the device cannula 102, and the second open lumen of the catheter 107.

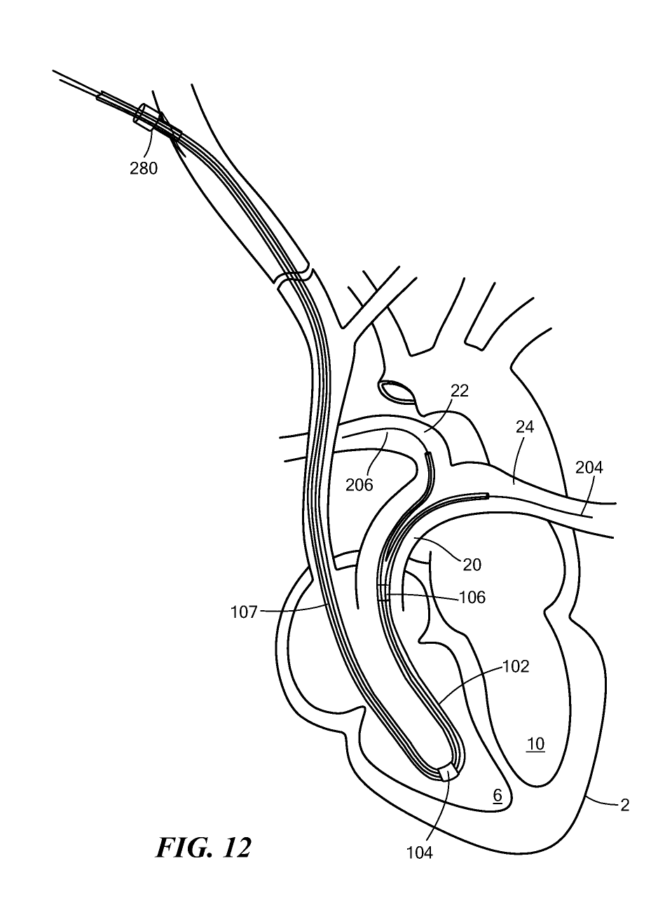

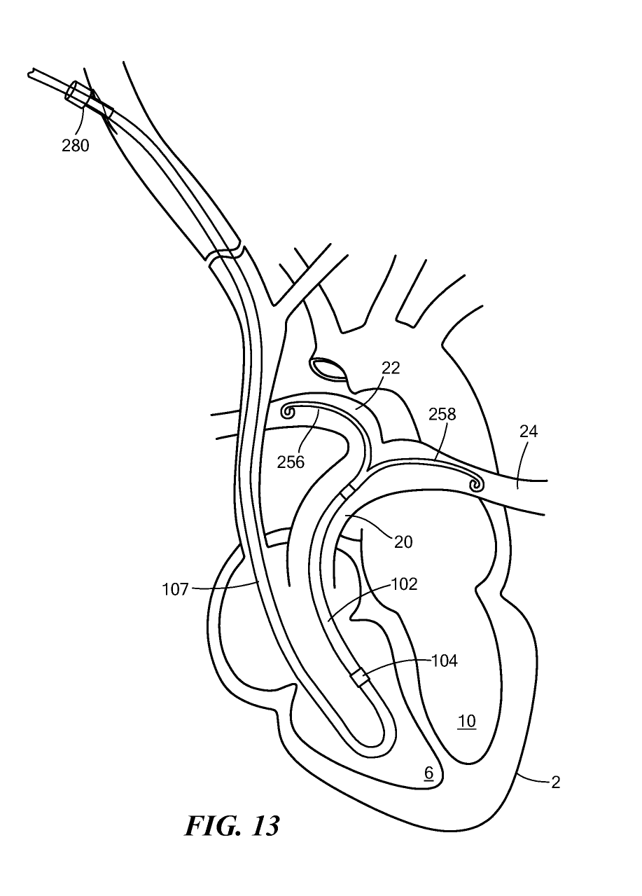

Referring to FIG. 12, the device 100 is then advanced along the first and second guide wires until the first tip portion 256 resides in the right pulmonary artery 22, and the second tip portion 258 resides in the left pulmonary artery 24. Then, the first and second guide wires 204, 206 are withdrawn from the respective the lumens to permit the first and second tip portions 256, 258 to support the PCAD device 100 and maintain its position within the pulmonary artery 20.

Referring to FIG. 13, the final deployed configuration of the device 100 is illustrated. Once the guide wires 204, 206 are withdrawn, treatment fluids can be provided to the respective blood vessels 22, 24 through one or both of the above described passageways. For example, an anti-clotting agent can be delivered to one or both of the right and left pulmonary arteries through the corresponding channels 260, 262 formed in the tip 250. Thus, the tip portions 256, 258 permit delivery of treatment fluids to targeted branches of a blood vessel in addition to serving as stabilizing support members for the device.

Although the method is described here as using the device 100 in an intra jugular approach, the device and method are not limited to this approach. For example, the device can be used in any approach in which it is deployed to the heart via either the superior vena cava or the inferior vena cava.

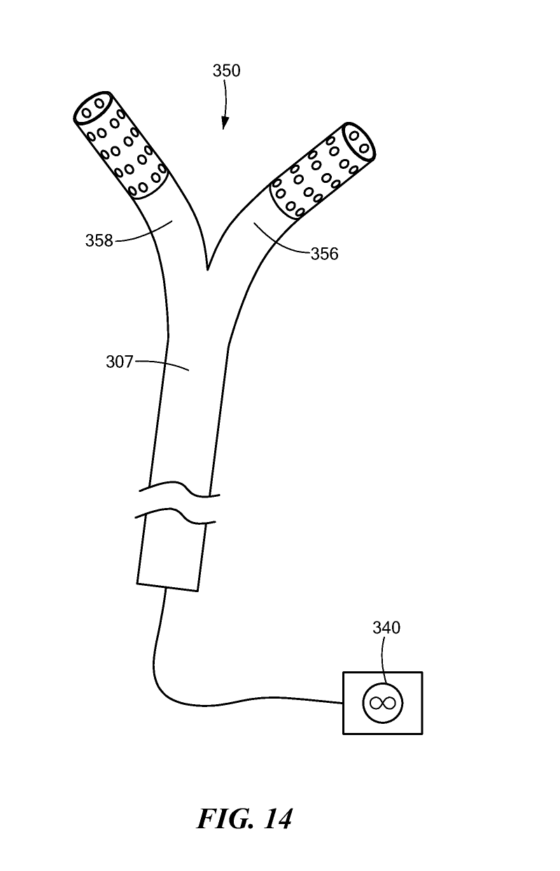

Referring to FIG. 14, although the tip 250 is illustrated herein as being disposed on an end of the cannular housing 102 of a pCAD of the type in which the fluid pump 104 is positioned within the body during use, it is not limited to use on this type of cardiac assist device. For example, this structure can be applied to a pCAD of the type in which the fluid pump is positioned outside the body. In this type of device, only a cannula 307 passes through the vessels to the heart 2, while the pumping portion 340 of the device is externally located. In this type of device, the cannula 307 is provided with a bifurcated tip 350. As in the previously described embodiment, the tip 350 includes a first tip portion 356 that can be placed in one branch of the vessel, and a second tip portion 358 that can be placed in the another branch of the vessel, whereby the cannula is maintained in a desired position within the main branch of the vessel.

Although the tip 250 is disclosed has having utility for stabilizing a catheter 102, 307 associated with a PCAD, the tip 250 is not limited to this application. For example, a dual-lumen bifurcated tip can be provided on leading ends of general use catheters for the purpose of maintaining a desired position of a catheter within a branched vessel.

In an alternative embodiment, the tip 250 may be configured to permit adjustment of the distance of the tip distal end 253 from the outlet end 106 of the device 100. For example, this may be accomplished by providing the tip as a separate member from the device 100 that is axially slideable along a passageway extending through the device 100 and catheter 107.

A selected illustrative embodiment of the invention is described above in some detail. It should be understood that only structures considered necessary for clarifying the present invention have been described herein. Other conventional structures, and those of ancillary and auxiliary components of the system, are assumed to be known and understood by those skilled in the art. Moreover, while a working example of the present invention has been described above, the present invention is not limited to the working example described above, but various design alterations may be carried out without departing from the present invention as set forth in the claims.

1-22. (canceled)

23. A percutaneous cardiac assist device comprising:
a fluid pump,
a tube configured to provide a passageway for fluid pumped by the fluid pump, the tube including:
a tube first end, and
a tube second end opposed to the tube first end,
wherein the tube second end is configured to serve as a fluid outlet from the pump, and
a bifurcated, flexible tip extends from the tube second end.

24. The device of claim 23, wherein the tube first end is configured to serve as an inlet to the fluid pump.

25. The device of claim 23, wherein the pump pumps the fluid axially through the passageway.

26. The device of claim 23, wherein the pump includes an impeller for pumping the fluid through the passageway.

27. The device of claim 23, wherein the passageway comprises a plurality of lumens, each lumen extending through at least a portion of the tube.

28. The device of claim 27, wherein the plurality of lumens includes a central lumen configured as a pump housing.

29. The device of claim 27, wherein the plurality of lumens includes a wiring lumen having electrical leads for controlling and operating the pump.

30. The device of claim 27, wherein the plurality of lumens includes a sensor lumen configured for providing fluid to one or more fluid pressure sensors.

31. The device of claim 23, wherein the tip further comprises:
a proximal end configured to secure to the tube second end, a bifurcated distal end opposed to the proximal end and including a first tip portion and a second tip portion that is detached from the first tip portion;
a first channel extending through the tip between the proximal end and a terminal end of the first tip portion; and
a second channel extending through the tip between the proximal end and a terminal end of the second tip portion.

32. The device of claim 31, wherein the tube includes a lumen that is in fluid communication with the first and second channels of the tip.

33. The device of claim 31, wherein the first and second channels are configured to receive a guide wire therethrough.

34. The device of claim 31, wherein each of the first tip portion and the second tip portion is configured to curl back on itself when a guide wire is not present within the respective first and second channels.

35. The device of claim 31, wherein the distal end of the tip is adjustable relative to the tube second end.

36. The device of claim 23, wherein the tip is generally Y shaped.

37. The device of claim 23, wherein the device is configured to be deployed to the heart via at least one of the superior vena cava and the inferior vena cava.

38. The device of claim 23, wherein the device is configured to be deployed to the heart via the jugular vein.

39. The device of claim 23, wherein the tip includes one or more fluid pressure sensors.

40. The device of claim 39, wherein the one or more fluid pressure sensors is disposed in the proximal end of the tip.

41. The device of claim 40, wherein the tube first end is configured for connecting to a catheter.

42. The device of claim 41, wherein the catheter is connected to a controller and a power supply.

43. A method of using a percutaneous assist device having a dual-lumened flexible tip, the leading end of the tip being bifurcated to form a first tip portion and a second tip portion that is detached from the first tip portion, each of the first and second tip portions including a respective lumen,
the method including the steps of
forming a percutaneous puncture in the jugular vein;
advancing a first guide wire through the puncture to the right pulmonary artery;
advancing a second guide wire through the puncture to the left pulmonary artery;
mounting the assist device on both the first and second guide wires such that the first guide wire extends through one respective lumen and the second guide wire extends through the other respective lumen;
advancing the assist device along the first and second guide wires until the first tip portion resides in the right pulmonary artery, and the second tip portion resides in the left pulmonary artery; and
withdrawing the guide wires from respective the lumens to permit the first and second tip portions to support the assist device within the pulmonary artery.

44. The method of claim 43, wherein the method further comprises the step of providing treatment fluids to the body through at least one of the lumens.
